(19) United States
(12) Patent Application Publication
(10) Pub. No.: US 2015/0290135 A1
(43) Pub. Date: Oct. 15, 2015

(54) PROCESS FOR MAKING AGGLOMERATES USING ACOUSTIC MIXING TECHNOLOGY

(71) Applicants: Sai Prasanth CHAMARTHY, Summit, NJ (US); Chinedu G. OREKIE, Summit, NJ (US); MERCK SHARP & DOHME CORP., Rahway, NJ (US)

(72) Inventors: Sai Prasanth Chamarthy, Hillsborough, NJ (US); Chinedu G. Orekie, Linden, NJ (US)

(73) Assignee: MERCK SHARP & DOHME CORP., RAHWAY, NJ (US)

(21) Appl. No.: 14/442,905

(22) PCT Filed: Nov. 12, 2013
(86) PCT No.: PCT/US13/69542

§ 371 Date: May 14, 2015

Related U.S. Application Data
(60) Provisional application No. 61/727,286, filed on Nov. 16, 2012.

(51) Int. Cl. A61K 9/16; A61K 31/58; A61K 9/00; A61K 31/56

(52) U.S. Cl. CPC A61K 9/1694; A61K 31/56; A61K 31/58; A61K 9/1623; A61K 9/16; A61K 9/0075

(57) ABSTRACT

Described herein is a process for preparing agglomerates comprising: (i) providing a dry powder mixture of one, two, or three active pharmaceutical agent(s), and at least one excipient; and (ii) applying acoustic energy to said dry powder mixture to form agglomerates.

Companies have developed dry powder inhaler (DPI) systems for administering powdered medications, such as those described in PCT International Publication No. WO 94/14492, which was published on Jul. 7, 1994 and is hereby incorporated by reference. Such inhaler systems are designed to meter out an exact dose of a powdered medication. DPI systems often utilize agglomerates or pellets which include an active pharmaceutical agent (APA) and optionally one or more excipients. One method of agglomerating APAs is described in PCT International Publication No. WO 95/09616, published on Apr. 13, 1995.

### BACKGROUND OF THE INVENTION

The conventional agglomerate manufacturing process includes the following steps: blending of micronized APAs and micronized lactose anhydrous NF to create a homogeneous powder blend using a v-blender, followed by agglomerating of the powder blend in a Ro-Tap® sieve shaker, and curing of the resultant agglomerates under controlled temperature and humidity. Subsequently, the resulting agglomerates may be placed into a reservoir in a dry powder inhaler (DPI) such as the TWISTHALER® device.

Agglomerates can be made by the methods described in U.S. Pat. No. 4,161,516, which are incorporated herein. Such methods may use certain binding materials, including water, for the production of agglomerates for oral inhalation. According to the processes described therein, prior to agglomeration, the moisture content of certain “self-agglomerating” or hygroscopic micronized APAs are elevated. After the micronized powder has been elevated to the desired water content level, it is agglomerated. Non-hygroscopic materials may be bound with more traditional binders as described therein. Similarly, WO 95/05805 discloses a process for forming agglomerates where a mixture of homogeneous micronized materials can be treated with water vapor to eliminate any convertible amorphous content which may destabilize at a later point. After treatment with water vapor, the now crystalline material is agglomerated.

While current DPI systems represent a significant advance in oral inhalation therapy, there are some circumstances in which problems remain. For instance, it has been found that some formulations with certain APAs or excipients, form poor agglomerates or may not form agglomerates at all. Problems with agglomerates or agglomerate formation will limit or prevent the use of an APA in a dry powder inhaler device.

Agglomeration problems often center on the properties of the APA and its ability to agglomerate. For example, certain APAs may not be “free-flowing”, may suffer from electrostatic charge problems, be too fluffy or exhibit an unacceptable degree of cohesive force. Furthermore, agglomeration problems may be related to recrystallization, micronization or higher load of an APA in the agglomerate so that an APA may not be able to form agglomerates. For instance, large doses of an APA may result in agglomerates with integrity problems and thereby preventing the APA from being used in a dry powder inhaler device. Conventional agglomerating processes may also be limited to using a single binder or carrier such as lactose. Other excipients/carriers may exhibit problems similar to those observed with problematic APAs.

Accordingly, it would be advantageous to provide a process for preparing agglomerates that overcome the issues with conventional agglomerate processing technologies. More particularly, it would be advantageous to provide a process for preparing agglomerates with high APA loads and varied excipient compositions.

### SUMMARY OF THE INVENTION

The process of the present invention addresses the above-mentioned unmet need present in the current processes for the preparation of agglomerates, and provides other advantages that will become apparent from the following detailed description. Thus, one aspect the present invention is a process for preparing agglomerates comprising: (i) providing a dry powder mixture of one, two, or three active pharmaceutical agent(s) (APA), and at least one excipient; (ii) applying acoustic energy to said dry powder mixture; and (iii) producing agglomerates from said dry powder mixture.

### DETAILED DESCRIPTION OF THE INVENTION

The instant invention provides a process for preparing agglomerates comprising: (i) providing a dry powder mixture of one, two, or three active pharmaceutical agent(s), and at least one excipient; and (ii) applying acoustic energy to said dry powder mixture to form agglomerates.

The instant invention provides a process for preparing agglomerates comprising: (i) providing a dry powder mixture of one or two active pharmaceutical agent(s), and at least one excipient; and (ii) applying acoustic energy to said dry powder mixture to form agglomerates.

The instant invention provides a process for preparing agglomerates comprising: (i) providing a dry powder mixture of one active pharmaceutical agent, and at least one excipient; and (ii) applying acoustic energy to said dry powder mixture to form agglomerates.

The instant invention provides a process for preparing agglomerates comprising: (i) providing a dry powder mixture of one active pharmaceutical agent, and two excipients; and (ii) applying acoustic energy to said dry powder mixture to form agglomerates.

The instant invention provides a process for preparing agglomerates comprising: (i) providing a dry powder mixture of one active pharmaceutical agent, and one excipient; and (ii) applying acoustic energy to said dry powder mixture to form agglomerates.

In an embodiment, the active pharmaceutical agent(s) are selected from corticosteroids, dissociated steroids, β-agonists, anticholinergics, leukotriene antagonists, spleen tyrosine kinase (Syk) inhibitors, Janus kinase (JAK) inhibitors, serotonergic agents, antibiotics, and inhalable proteins or peptides. In another embodiment, the active pharmaceutical agent(s) are selected from glycopyrrolate, ciclesonide, indacaterol, tiotropium, mometasone furoate, beclomethasone dipropionate, budesonide, fluticasone, dexamethasone, flunisolide, triamcinolone, salbutamol, albuterol, terbutaline, salmeterol, bitolterol, ipratropium bromide, oxitropium bromide, sodium cromoglycate, nedocromil sodium, montelukast, zafirlukast, pranlukast, formoterol, eformoterol, bambuterol, fenoterol, clenbuterol, procaterol, broxaterol, (22R)-6α,9α-difluoro-11β,21-dihydroxy-16α,17α-propylmet hylenedioxy-4-pregnen-3,20-dione, TA-2005, tipredane, insulin, interferons, calcitonins, parathyroid hormones, sumatriptan, rizatriptan, naratriptan, zolmitriptan, eletriptan, almotriptan, frovatriptan, avitriptan, tobromycin, and granulocyte colony-stimulating factor. In another embodiment, the active pharmaceutical agent(s) are selected from glycopyrrolate, ciclesonide, indacaterol, tiotropium, mometasone furoate, budesonide, fluticasone, triamcinolone, salmeterol, montelukast, zafirlukast, pranlukast, rizatriptan, tobromycin, and formoterol. In another embodiment, the active pharmaceutical agent is mometasone furoate.

In another embodiment, the excipient is selected from polyhydroxy aldehydes, and polyhydroxy ketones. Preferred polyhydroxy aldehydes and polyhydroxy ketones include hydrated and anhydrous saccharides selected from lactose, glucose, fructose, galactose, trehalose, sucrose, maltose, raffinose, mannitol, melezitose, starch, xylitol, mannitol, myoinositol, their derivatives, and the like. In another embodiment, the at least one excipient is selected from lactose, sorbitol, xylitol, and mannitol. Preferred polyhydroxy aldehydes and polyhydroxy ketones include hydrated and anhydrous saccharides selected from glucose, fructose, galactose, trehalose, sucrose, maltose, raffinose, mannitol, melezitose, starch, xylitol, mannitol, myoinositol, their derivatives, and the like. In another embodiment, the at least one excipient is selected from sorbitol, xylitol, and mannitol.

In another embodiment, said acoustic energy is applied at a low frequency. In another embodiment, said low frequency ranges from about 10 Hertz to about 1000 Hertz. In another embodiment, said low frequency ranges from about 50 to about 200 Hertz. In another embodiment, said low frequency ranges from about 58 Hertz to about 64 Hertz.

In another embodiment, said acoustic energy is applied as a standing wave supplying a linear acceleration from about 10 times to about 100 times the force of gravity for about 5 to about 30 minutes. In another embodiment, said linear acceleration is from about 40 times to about 100 times the force of gravity for about 10 minutes.

In another embodiment, said acoustic energy is supplied by a resonance acoustic mixing device.

In another embodiment, said acoustic energy is supplied by the Resodyn™ acoustic mixer.

In another embodiment, the agglomerates are formed utilizing a sieve shaker.

In another embodiment, said sieve shaker is a Ro-Tap® sieve shaker.

In another embodiment, the agglomerates produced by the instant process have a bulk density of between about 0.2 and about 0.4 g/cm³. In another embodiment, the agglomerates produced by the instant invention have a bulk density of between about 0.23 and about 0.38 g/cm³.

In another embodiment, the agglomerates produced by the instant process contain at least about 40 weight percent of excipient.

In another embodiment, said dry powder mixture may form agglomerates by processing one, two, or three active pharmaceutical agent(s) with one or more excipients.

In another embodiment, said dry powder mixture may form agglomerates by processing a single active pharmaceutical agent and at least one excipient and separately processing a different active pharmaceutical agent with at least one excipient to form agglomerates and blending the separate agglomerations together to obtain a final agglomeration.

Other embodiments of the present invention provide for a dosage form useful for administration by oral inhalation therapy comprising agglomerates, wherein the active pharmaceutical agent(s) and at least one excipient have an average particle size of about 10 μm or less and being provided in a weight ratio of between 100:1 to 1:500, the agglomerates having an average size of between about 300 and about 700 μm, a bulk density of between about 0.2 and about 0.4 g/cm³ and a crush strength of between about 200 mg and about 1500 mg.

Additional embodiments of the present invention provide for a medicinal product comprising a dry powder inhaler and the agglomerates. Additional embodiments of the present invention provide for a method of producing agglomerates comprising blending a powder comprising one, two or three active pharmaceutical agent(s) and at least one excipient; subjecting the blended powder to an acoustic mixing device and then agglomerating the powder into agglomerates. Other embodiments provide for a pharmaceutical product comprising a dry powder inhaler and the agglomerates as produced by this method.

Most preferably, in accordance with the present invention, the active pharmaceutical agent(s) is/are a material capable of being administered in a dry powder form to the respiratory system, including the lungs. For example, an active pharmaceutical agent(s) in accordance with the present invention could be administered so that it is absorbed through the lungs. More preferably, however, the active pharmaceutical agent(s) is/are a powder which is effective to treat some condition of the lungs or respiratory system directly and/or topically.

It is important that the process produce agglomerates ranging in size from between about 100 to about 1500 μm. The agglomerates generally have an average size of between about 300 and about 1,000 μm. More preferably, the agglomerates have an average size of between about 400 and about 700 μm. Most preferably, the agglomerates will have an average size of between about 500 and about 600 μm. The resulting agglomerates will also have a bulk density which ranges from between about 0.2 to about 0.4 g/cm³ and more preferably, between about 0.29 to about 0.38 g/cm³. Most preferably, the agglomerates will have a bulk density which ranges from between about 0.31 to about 0.36 g/cm³.

It is also important to the dosing of the active pharmaceutical agent(s) that the agglomeration process yields a relatively tight particle size distribution. In this context, particle size refers to the size of the agglomerates. Preferably, no more than about 10% of the agglomerates are 75% smaller or 75% larger than the mean or target agglomerate size. Thus, for a desired agglomerate of 300 μm, no more than about 10% of the agglomerates will be smaller than about 100 μm or larger than about 500 μm.

Acoustic mixers, for example, the Resodyn™ acoustic mixer, are commercially available. This technology has been described, for example, in U.S. Pat. No. 7,188,993 to Howe et al., and employs linear displacement to introduce a standing linear acoustic wave into a medium, for example, gas, liquid or solid, residing within a container affixed to the device. Preparation of admixtures comprising energetic or shock-sensitive materials has been described using acoustic mixing, for example, in Published U.S. Patent Application 2010/0294113 (McPherson). The blending of dissimilar powders has also been described, for example, the blending of sand with fumed silica using an acoustic mixer (Resodyn™ marketing literature).

A resonance acoustic mixing unit has been used for intimate processing, for example, mixing a plurality of fluids, e.g., intimately mixing a gas in a liquid, or a liquid in another liquid, or more than two phases. One application is the mixing and dispersion of solids in liquids, in particular hard to wet solids and small particles. Other applications include preparing emulsions for chemical and pharmaceutical applications, gasifying liquids for purification and for chemical reactions, accelerating physical and chemical reactions, and suspending fine particles in fluids. The fluids to which reference is made herein may or may not include entrained solid particles. However, resonance acoustic mixing has never been utilized as done in various aspects of the present invention. Specifically, resonance acoustic mixing is utilized to mix the dry powders prior to the agglomeration stage of the process. Utilization of the resonance acoustic mixer allows the various aspects of the present invention to overcome some of the issues of conventional agglomeration process technology. Surprisingly, the various aspects of the present invention provide for agglomerates that can hold a higher APA load as well as provide agglomerates of certain ‘difficult to handle’ APAs, such as dissociated steroids, that do not agglomerate or have agglomeration issues using conventional blending technology during the agglomeration process.

As the term is used herein, acoustic energy is linear or spherical energy propagation through a tangible medium which is within the frequency range of about 10 hertz to about 20,000 hertz. In some embodiments of the process of the present invention, it is preferred to employ linear acoustic energy at a frequency of from about 10 Hertz up to about 1000 Hertz, more preferably the acoustic energy is supplied at a frequency of about 50 to about 200 Hertz, and most preferably the acoustic energy is supplied at a frequency of about 58 to about 64 Hertz. It will be appreciated that in processes of the invention, in accordance with known principles, the exact frequency will be selected to provide a standing wave in the dry powder mixture. The frequency required to achieve a standing wave will vary according to known principles depending upon the nature and the dimensions of the dry powder to which acoustic energy is applied.

Acoustic energy can be supplied to an admixture using any known source; however, in general it is preferred to supply the energy by cyclic linear displacement of a container filled with the admixture. In processes of the invention, preferably the acoustic energy supplied by linear displacement exerts between about 10 times G-force (where “G” is the force of gravity) and about 100 times G-force. Although it will be appreciated that numerous mechanical or electronic transducer arrangements can be utilized to supply the cyclic linear displacement required to generate the desired G-force within the desired frequency range, one example of commercially available equipment suitable for supplying the necessary acoustic energy is the Resodyn™ acoustic mixer (Resodyn Acoustic Mixers, Inc.), which makes equipment available in a range of capacities from bench-scale to multi-kilogram capacity. During blending, the entire system (the RAM machine components with the material being mixed) is maintained at resonance, which facilitates very efficient energy transfer, from the machine to the mixing material (the formulation in this case). The material is subjected to accelerations up to 100 times the force of gravity. This results in fluidization and randomization of the material with in the mixing container.

As mentioned above, it was previously known that an acoustic mixer such as a Resodyn™ acoustic mixer could be used to efficiently mix dry powder materials, however, acoustic mixing has not been previously employed to prepare agglomerates from bulk powdered solid materials. Surprisingly, the inventors have found that the use of acoustic energy to prepare agglomerates provides an agglomerate product using materials which could not be produced using the traditional manufacturing process.

“Active pharmaceutical agent(s) (APA)”, means any substance intended to be used in the manufacture of a drug product that becomes an active ingredient in the drug product. Active pharmaceutical agents include, but are not limited to corticosteroids, dissociated steroids, β-agonists, anticholinergics, leukotriene antagonists, spleen tyrosine kinase (Syk) inhibitors, Janus kinase (JAK) inhibitors, serotonergic agents, antibiotics, and inhalable proteins or peptides. The active pharmaceutical agent(s) may comprise at least one member selected from the group consisting of: glycopyrrolate, ciclesonide, indacaterol, tiotropium, mometasone furoate, beclomethasone dipropionate, budesonide, fluticasone, dexamethasone, flunisolide, triamcinolone, salbutamol, albuterol, terbutaline, salmeterol, bitolterol, ipratropium bromide, oxitropium bromide, sodium cromoglycate, nedocromil sodium, montelukast, zafirlukast, pranlukast, formoterol, eformoterol, bambuterol, fenoterol, clenbuterol, procaterol, broxaterol, (22R)-6α,9α-difluoro-11β,21-dihydroxy-16α,17α-propylmet hylenedioxy-4-pregnen-3,20-dione, TA-2005, tipredane, insulin, interferons, calcitonins, parathyroid hormones, sumatriptan, rizatriptan, naratriptan, zolmitriptan, eletriptan, almotriptan, frovatriptan, avitriptan, tobromycin, and granulocyte colony-stimulating factor. Another embodiment of the active pharmaceutical agent(s) may comprise at least one or more member selected from the group consisting of: glycopyrrolate, ciclesonide, indacaterol, tiotropium, mometasone furoate; budesonide; fluticasone; triamcinolone; salmeterol; montelukast; zafirlukast; pranlukast; rizatriptan; tobromycin; and formoterol. Additionally, it is contemplated that agglomerates could be formed with 1, 2, or 3 APAs. Examples of such combinations are: mometasone furoate and tiotropium; mometasone furoate and glycopyrrolate; mometasone furoate, glycopyrrolate, and formoterol; mometasone furoate and inhaled spleen tyrosine kinase (Syk) inhibitors; and salmeterol and fluticasone.

“Agglomerate” means a bound mass of small particles. Agglomeration refers to the process of producing agglomerates. Agglomerates include at least one first material and at least one solid binder. The first material, in accordance with the present invention can be anything as, indeed, the present invention can be used broadly to make free-flowing agglomerates for any application including, medicine, cosmetics, food and flavoring, and the like. However, preferably, the first material is an active pharmaceutical agent(s) which is to be administered to a patient in need of some course of treatment. The active pharmaceutical agent(s) may be administered prophylactically as a preventative or during the course of a medical condition as a treatment or cure. Suitable agglomerates refer to agglomerates that may be used in a dry powder inhaler system, such as the TWISTHALER® used in ASMANEX.

“Dry powdered mixture” means a mixture of finely divided active pharmaceutical agents and/or chemicals in dry form.

“Excipients” means any inert substance in a pharmaceutical dosage form that is not an active pharmaceutical agent. Excipients include binders, lubricants, diluents, disintegrants, coatings, barrier layer components, glidants, and other components. added to a drug to give suitable consistency or form to the drug product. Excipients include but are not limited to lactose, sorbitol, xylitol, and mannitol.

The present invention will be further understood from the following examples, which are meant to illustrate rather than limit the invention.

### EXAMPLES

During formulation work, alternate excipients to lactose (such as xylitol, mannitol, and sorbitol) were explored for making agglomerates. These excipients were micronized such that their median size was less than 2 microns. These alternative excipients were used to make mometasone furoate (MF) formulations. The conventional agglomerate manufacturing process as described above was used and is also described in U.S. Pat. Nos. 6,503,537 and 6,623,760, which are both incorporated herein. It was observed that none of these formulations made with alternative excipients agglomerated to any appreciable degree using the conventional process.

Agglomerates from all these alternate excipient batches, at the same formulation composition used for the conventional process (Table 2), were successfully produced by an alternate manufacturing process. This modified process used a resonance acoustic mixer (RAM) as the blending apparatus instead of a V-blender. The rest of the manufacturing steps were identical to that of the conventional manufacturing process. The formulations were mixed using RAM at up to 100 times the force of gravity. The mixed powders were then transferred to the agglomeration stage of the conventional agglomerate manufacturing process. Surprisingly, the acoustically blended powder mixtures with the alternate carriers agglomerated. This experience was notably quite different from previous work with the same materials using the conventional DPI manufacturing process. The agglomerates recovered per each combination of APA and alternate excipients/carriers were characterized for their physical characteristics (particle size distribution and density) and summary data is presented in Table 2 below. This unexpected and surprising result is an effective means for influencing the agglomeration tendency of formulations. This technology is expected to be a versatile technique for current and future development projects, which are intended to deliver therapeutic agents to the patient in the form of agglomerates.

The following examples demonstrate the advantages available from the process of the present invention and the agglomerates provided according to the present invention.

In the following examples, agglomerates of the invention were prepared with acoustic power supplied using a Resodyn™ Resonant Acoustic Mixer® (LabRAM™) and the indicated power settings. In the examples, comparative agglomerate samples were prepared using a V-blender apparatus equipped with an intensification bar.

Example agglomerates were evaluated for particle size and particle size distribution using Sympatec laser diffraction particle size analyzer equipped with a GRADIS (gravimetric dispersion) dry powder disperser and a vibratory feeder. Results reported for particle size analysis (D50 and D90) have their ordinary meaning as understood in the art of particle-size analysis. Unless otherwise indicated, values for D50 and D90 are reported in micrometers (μm).

As used in the examples, all excipients are articles of commerce unless otherwise noted.

### Example 1

### Preparation of Agglomerates Using an Alternative Process for an APA that Did not Form Agglomerates Using the Traditional Process

Three batches of dissociated steroid APA, were manufactured (APA Lot Numbers A, B, and C) as a part of process optimization studies for the APA manufacture. These APA batches of dissociated steroid were subsequently used to formulate the higher active pharmaceutical agent load dissociated steroid formulations (1000 mcg/inhalation, formulation details Table 1), using the typical mixing and agglomeration process which includes blending of micronized APAs and micronized excipients to create a homogeneous powder blend using a v-blender, followed by agglomerating of the powder blend in a Ro-Tap® sieve shaker, and curing of the resultant agglomerates under controlled temperature and humidity. This traditional process is also described in U.S. Pat. No. 6,503,537 and U.S. Pat. No. 6,623,760, which are both incorporated herein. None of these batches produced agglomerates in the Ro-Tap®.

TABLE 1
Agglomerate particle size distributions for Dissociated Steroid 1000
mcg/inhalation batches. (40% w/w Compound A, 60% w/w lactose)

| APA Lot | | Agglomerate PSD (μm) | | |
|---|---|---|---|---|
| No. | Blending Process | X10 | X50 | X90 |
| A | Traditional Blending | No agglomerates formed | | |
| | Process | | | |
| B | Traditional Blending | No agglomerates formed | | |
| | Process | | | |
| C | Traditional Blending | No agglomerates formed | | |
| | Process | | | |
| A | RAM (10 minutes) | 264.29 | 653.75 | 930.43 |
| A | RAM (20 minutes) | 294.19 | 482.03 | 672.97 |
| B | RAM (10 minutes) | 210.79 | 488.77 | 723.34 |
| B | RAM (15 minutes) | 287.84 | 523.41 | 713.76 |
| B | RAM (20 minutes) | 240.47 | 509.10 | 709.07 |
| C | RAM (20 minutes) | 218.18 | 469.62 | 678.04 |

Agglomerates from all these APA batches, at the same formulation composition, were successfully produced by an alternate manufacturing process (Table 1). This modified process used a resonance acoustic mixer (RAM) as the blending apparatus instead of the V-blender. The rest of the manufacturing steps were identical to that of the typical manufacturing process and is described below. The particle size distributions of the agglomerates were measured using a Sympatec laser diffraction particle size analyzer equipped with a GRADIS (gravimetric dispersion) dry powder disperser and a vibratory feeder.

Resonance acoustic mixing is a technology that relies on low frequency (58-64 Hz), high-intensity acoustic energy. The 50 g of material to be blended was metered into a container and firmly attached to a LabRAM™ machine. The LabRAM™ acoustic mixer was run at 100% power for ten minutes. Our results demonstrate that this blending process induces the formulation to agglomerate in the Ro-Tap®. The exact nature of this phenomenon is currently under investigation.

After acoustic mixing, the powder blend is then agglomerated using a Ro-Tap® sieve shaker (Tyler RX-30). The Ro-Tap® used is a 12 in. diameter (approximately 30 cm) sieve shaker kept under the same temperature and humidity conditions as the blender (70±5° F. {21±3° C.} and 20%±5% RH). An assembly of four sets of #30 mesh screen (ASTM)/pan combinations is used. Powder blend is poured onto the #30 mesh screen. Four sets are stacked on top of each other and the top screen is fitted with a cover. The entire set is then placed on the sieve shaker. The set is then subjected to simultaneous tapping and rotation by the Ro-Tap®. The tapping motion forces the powder through the mesh onto the pan, where the agglomerates are formed with an eccentric rotation. After the Ro-Tap® run, the resulting agglomerates are manually sieved through a #20 mesh screen and placed inside a curing chamber (25° C./50% RH) for a period of 24 hours before final storage and filling.

### Example 2

### Preparation of Agglomerates Using an Alternative Process for Various Excipients that Did not Form Agglomerates Using the Traditional Process

During formulation work, alternate excipients to lactose (such as xylitol, mannitol, and sorbitol) were explored. These excipients were micronized such that their median size was less than 2 microns. These excipients were used to make mometasone furoate (MF) formulations. The conventional agglomerate manufacturing process which includes blending of micronized APAs and micronized excipients to create a homogeneous powder blend using a v-blender, followed by agglomerating of the powder blend in a Ro-Tap® sieve shaker, and curing of the resultant agglomerates under controlled temperature and humidity was used to manufacture the batches. It was observed that none of these formulations agglomerated to any appreciable degree (Batch Nos. A, B, and C).

TABLE 2
Physical Characteristics of Agglomerates from alternate
excipients and processes for mometasone furoate 200 mcg/inhalation
(where MF: 14.7% w/w, excipient: 85.3% w/w)

| | | | Agglomerate | | Agglomerate Particle | | |
|---|---|---|---|---|---|---|---|
| Batch | | Blending | Density (g/mL) | | Size Distribution (μm) | | |
| No. | Excipient | Process | Bulk | Tap | X10 | X50 | X90 |
| A | Sorbitol | Traditional | No agglomerates formed | | | | |
| | | Process | | | | | |
| B | Xylitol | Traditional | No agglomerates formed | | | | |
| | | Process | | | | | |
| C | Mannitol | Traditional | No agglomerates formed | | | | |
| | (Placebo | Process | | | | | |
| | batch) | | | | | | |
| D | Lactose | RAM | 0.34 | 0.35 | 297.55 | 487.01 | 661.40 |
| | | 10 minutes | | | | | |
| E | Sorbitol | RAM | 0.25 | 0.26 | 305.30 | 461.52 | 608.24 |
| | | 10 minutes | | | | | |
| F | Xylitol | RAM | 0.26 | 0.27 | 445.37 | 608.57 | 754.90 |
| | | 10 minutes | | | | | |
| G | Mannitol | RAM | 0.27 | 0.30 | 294.98 | 441.84 | 597.89 |
| | | 10 minutes | | | | | |
| H | Lactose | Traditional | 0.31 | 0.33 | 306.26 | 477.45 | 661.36 |
| | | Process | | | | | |

Agglomerates from all these alternate excipient batches, at the same formulation composition (Table 2), were successfully produced by an alternate manufacturing process. This modified process used a resonance acoustic mixer (RAM) as the blending apparatus instead of the V-blender. The rest of the manufacturing steps were identical to that of the typical manufacturing process. The 50 g formulations were mixed on a LabRAM™ acoustic mixing unit at 100% power for ten minutes. The mixed powders were then transferred to the agglomeration stage of the conventional agglomerate manufacturing process. The bulk and tap density and particle size distribution of each agglomeration was measured. The bulk density was determined by transferring between 9 to 10 mL of agglomerates into a 10 mL graduated cylinder, reading the aerated/uncompacted volume and mass of agglomerates transferred, and calculating the density as: mass(g)/volume(mL). Tap density was then determined by twice tapping the cylinder, reading the new settled volume, and calculating the density as: mass(g)/volume(mL). The particle size distributions of the agglomerates were measured using a Sympatec laser diffraction particle size analyzer equipped with a GRADIS (gravimetric dispersion) dry powder disperser and a vibratory feeder.

Surprisingly, the powder mixtures with the alternate carriers agglomerated when mixed with an acoustic mixer. This experience was notably quite different from previous work with the same materials using the conventional DPI manufacturing process. The agglomerates recovered per each combination of APA and alternate excipients/carriers were characterized for their physical characteristics (particle size distribution and density) and summary data is presented in Table 2 above. This unexpected and surprising result is an effective means for influencing the agglomeration tendency of formulations.

We claim:

1. A process for preparing agglomerates comprising: (i) providing a dry powder mixture of one, two, or three active pharmaceutical agent(s), and at least one excipient; and (ii) applying acoustic energy to said dry powder mixture to form agglomerates.

2. The process of claim 1, wherein said active pharmaceutical agent(s) active pharmaceutical agent(s) are selected from corticosteroids, dissociated steroids, β-agonists, anticholinergics, leukotriene antagonists, spleen tyrosine kinase (Syk) inhibitors, Janus kinase (JAK) inhibitors, serotonergic agents, antibiotics, and inhalable proteins or peptides.

3. The process of claim 2, wherein said active pharmaceutical agent(s) are selected from glycopyrrolate, ciclesonide, indacaterol, tiotropium, mometasone furoate, beclomethasone dipropionate, budesonide, fluticasone, dexamethasone, flunisolide, triamcinolone, salbutamol, albuterol, terbutaline, salmeterol, bitolterol, ipratropium bromide, oxitropium bromide, sodium cromoglycate, nedocromil sodium, montelukast, zafirlukast, pranlukast, formoterol, eformoterol, bambuterol, fenoterol, clenbuterol, procaterol, broxaterol, (22R)-6α,9α-difluoro-11β,21-dihydroxy-16α,17α-propylmet hylenedioxy-4-pregnen-3,20-dione, TA-2005, tipredane, insulin, interferons, calcitonins, parathyroid hormones, sumatriptan, rizatriptan, naratriptan, zolmitriptan, eletriptan, almotriptan, frovatriptan, avitriptan, tobromycin, and granulocyte colony-stimulating factor.

4. The process of claim 3, wherein said active pharmaceutical agent(s) are selected from glycopyrrolate, ciclesonide, indacaterol, tiotropium, mometasone furoate, budesonide, fluticasone, triamcinolone, salmeterol, montelukast, zafirlukast, pranlukast, rizatriptan, tobromycin, and formoterol.

5. The process of claim 1, wherein said excipient is selected from polyhydroxy aldehydes and polyhydroxy ketones.

6. The process of claim 5, wherein said excipient is selected from lactose, glucose, fructose, galactose, trehalose, sucrose, maltose, raffinose, mannitol, melezitose, starch, xylitol, mannitol, and myoinositol.

7. The process of claim 6, wherein said excipient is selected from lactose, xylitol, mannitol, and sorbitol.

8. The process of claim 1, wherein said acoustic energy is low frequency.

9. The process of claim 8, wherein said low frequency ranges from about 10 Hertz to about 1000 Hertz.

10. The process of claim 9, wherein said low frequency ranges from about 50 Hertz to about 200 Hertz.

11. The process of claim 10, wherein said low frequency ranges from about 58 Hertz to about 64 Hertz.

12. The process of claim 1, wherein said acoustic energy is a standing wave supplying a linear acceleration from about 9 times to about 100 times the force of gravity for about 5 to about 30 minutes.

13. The process of claim 12, wherein said linear acceleration is from about 40 times to about 100 times the force of gravity for 10 minutes.

14. The process of claim 1, wherein said acoustic energy is supplied by a resonance acoustic mixing device.

15. A pharmaceutical product comprising agglomerates produced by the process of claim 1.

16. A pharmaceutical product comprising a dry powder inhaler and agglomerates as produced by the process of claim 1.
